(19) United States
(12) Patent Application Publication
(10) Pub. No.: US 2010/0331628 A1
(43) Pub. Date: Dec. 30, 2010

(54) Wireless Monitoring and Control of Medical Devices

(75) Inventors: Sailesh Merchant, Macungie, PA (US); Kouros Azimi, Center Valley, PA (US); Mohammad Mobin, Orefield, PA (US)

Correspondence Address: IP Legal Services, 1500 East Lancaster Avenue, Suite 200, P.O. Box 1027, Paoli, PA, 19301, US

(73) Assignee: LSI Corporation

(21) Appl. No.: 12/493,608

(22) Filed: Jun. 29, 2009

(51) Int. Cl. A61B 5/00; G08B 23/00

(52) U.S. Cl. 600/300; 340/573.1

(57) ABSTRACT

In described embodiments, a data collection device receives signals from one or more patient monitoring devices, the signals representing patient data and other vital signs measured at a patient. The data collection device employs statistical quality algorithms to track irregular behavior and out-of-bound events, the behavior and events either being pre-set, adaptively set, or otherwise defined within pre-determined limits. The data collection device communicates alerting signals to a caregiver's handheld device when the irregular behavior and out-of-bound events occur. The alerting signals contain information related to the patient data, irregular behavior and out-of-bound events, thereby allowing a caregiver to take appropriate action.

### BACKGROUND OF THE INVENTION

1. Field of the Invention

The present invention relates to wireless communications, and, in particular, to wireless monitoring and control of medical devices.

2. Description of the Related Art

The medical industry employs many different types of patient monitoring devices, often to monitor vital signs of, for example, a human or other animal patient. Sensors are connected to a patient to monitor such vital signs as oxygen content, heart rate, blood pressure, temperature and the like. Such monitoring can take place while the patient is awake, or under anesthetic, such as during surgery. Further, either pre- or post-surgery, the patient might be monitored locally by various devices, but the monitoring devices might only be occasionally checked directly by a caregiver.

During a monitoring, one or more of the patient's vital signs might exhibit irregular behavior or experience and out-of-bound event (i.e., some monitored quantity exceeded a threshold). At the occurrence of such event, the monitoring device might sound an audio and/or visual alarm, requiring a caregiver to notice and respond to the alarm. A caregiver may or may not be able to respond to such alarm directly with respect to the patient. For example, if a nurse detects and acknowledges the alarm, the nurse might still be required to summon a doctor to interpret the circumstances surrounding the alarm. This situation might result in delay of the proper caregiver's ability to take appropriate action to the irregular behavior or out-of-bound event, possibly causing harm to the patient.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention allows for patient monitoring by collecting, with a data collection device (DCD) coupled to one or more monitoring devices through corresponding links, signals from one or more monitoring devices, wherein each signal represents patient data collected by one or more sensors coupled to a patient. Each signal is processed signal in accordance with one or more algorithms and the DCD generates at least one of a vital sign (VS) signal and an alerting signal for each signal based on the one or more algorithms for irregular behavior or an out-of-bound event with the corresponding signal. A portable communication device (PCD) in wireless communication with the DCD receives the VS signal and the alerting signal and provides information associated with the VS signal and the alerting signal to a caregiver.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements.

FIG. 1 shows a block diagram of a medical monitoring system operating in accordance with an exemplary embodiment of the present invention;

FIG. 2 shows a handheld monitor/alert device as might be in communication with the data collection device of FIG. 1; and

FIG. 3 shows an exemplary graph of data points versus thresholds as might be employed by the data collection device of FIG. 1 in accordance with statistical quality control.

### DETAILED DESCRIPTION

In described embodiments, a data collection device receives signals from one or more patient monitoring devices, the signals representing patient data measured at a patient. The data collection device employs statistical quality algorithms to track irregular behavior and out-of-bound event thresholds, the behavior and event thresholds either being pre-set, adaptively set, or otherwise defined within pre-determined limits. The data collection device communicates alerting signals to a caregiver's handheld device when the irregular behavior and out-of-bound event thresholds occur. The alerting signals contain information related to the patient data, irregular behavior and out-of-bound event thresholds, thereby allowing a caregiver to take appropriate action.

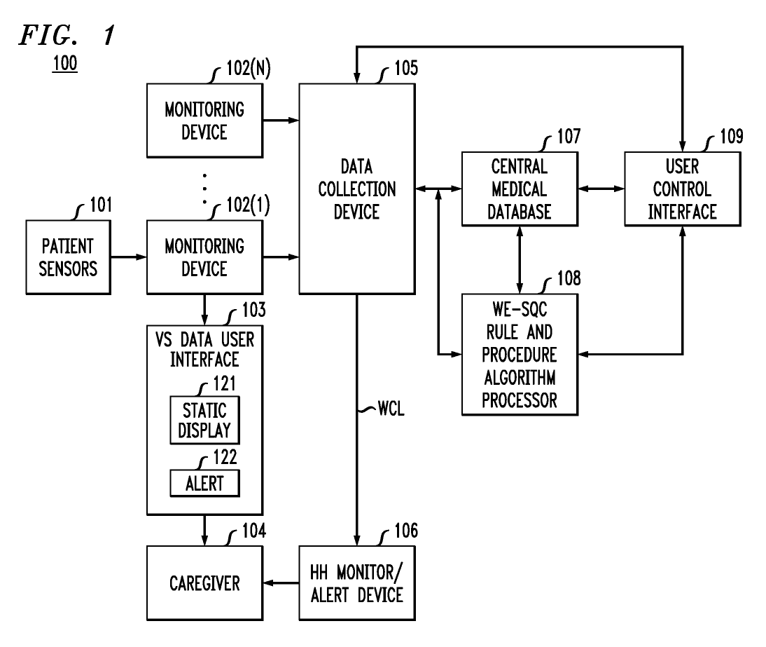

FIG. 1 shows a block diagram of a medical monitoring system 100 operating in accordance with an exemplary embodiment of the present invention. System 100 includes patient sensors 101 that are connected to a patient, which patient might be tended to by caregiver 104. Caregiver 104 might be a doctor, nurse, veterinarian, or other trained medical technician. Patient sensors 101 might be employed by a medical facility to monitor vital signs, such as oxygen content, heart rate, blood pressure, temperature and the like for use by caregiver 104. Patient sensors 101 are coupled to corresponding monitoring device 102(1), which monitors the patient's sensors to detect values for various vital signs. As shown in FIG. 1, system 100 might include more than one monitoring device, shown as monitoring devices 102(1) through 102(N). Depending on the medical facility, a single monitoring device might be employed for each patient, or a monitoring device might be employed for monitoring sensors of multiple patients.

Monitoring device 102(1) collects the raw sensor information related to vital signs and processes it for use by medical personnel. Consequently, monitoring device 102 might be coupled to vital sign (VS) data user interface 103 having status display 121 and alert module 122. Status display 121 might comprise a LCD or CRT display, or might be a printer, that shows collected VS information. Alert module 122 includes visual and/or audible alarm(s).

When monitoring device 102 processes sensor data corresponding to a VS, monitoring device 102 might detect irregular behavior or an out-of-bound event when a monitored quantity exceeds a threshold. When monitoring device 102 detects irregular behavior or an out-of-bound event, the detected condition is transmitted to VS data user interface 103, which indicates the detected condition to caregiver 104 through static display 121, and might employ visual and/or audible alarm(s) of alert module 122 to attract attention of caregiver 104. However, caregiver 104 might only receive information from VS data user interface 103 when caregiver 104 is co-located with VS data user interface 103, which is typically near patient sensors 101 (and, consequently, the patient).

Monitoring devices 102(1) through 102( ) are coupled to data collection device (DCD) 105. DCD 105, as described further subsequently, collects and processes information from monitoring device 105 for one or more patients (passive mode or active mode). Monitoring devices 102(1) through 102(N) might be coupled to DCD 105 through a wired connection, or might be coupled to DCD 105 through a wireless connection. Such wireless connection between one or more of monitoring devices 102(1) through 102( ) and DCD 105 might advantageously be employed to allow for monitoring of information collected by sensors implanted within the patient. Here, the term “passive mode” relates to sensors automatically collecting information when coupled to a patient without affirmative action (such as counting a pulse), while “active mode” relates to the sensor affirmatively applying a test to the patient (such as an electrical impulse to then measure muscle reaction) and waiting for a response.

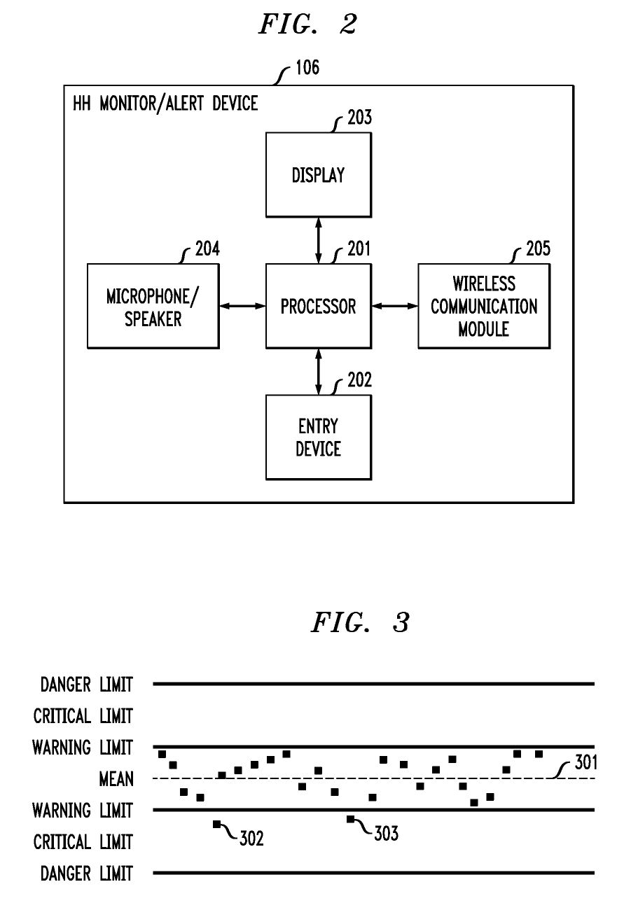

DCD 105 is also coupled to a portable communication device (PCD) that has data processing, alerting, and, optionally, display capabilities. The PCD might be implemented in any number of ways, such as in a device similar to a wireless remote phone (e.g., iPhone), intelligent PDA, or similar device. A PCD is shown in FIGS. 1 and 2 as handheld (HH) monitor/alert device 106 coupled to DCD 105 through wireless communication link (WCL). WCL might be implemented in any number of varying ways in the art of communication, such as in accordance with WiFi (802.11), WiMax (802.16), and personal area network (PAN) (802.15) standards, such as low frequency radio, or such as 2.5 and 3G wireless telephony standards. Preferred embodiments of the present invention employ wireless communication in accordance with the 802.11 family of standards. HH monitor/alert device 106 is employed by caregiver 104 to remotely monitor a patient, and is designed to be carried or in near proximity to caregiver 104 when caregiver 104 is not in proximity to the patient.

FIG. 2 shows an exemplary handheld monitor/alert device 106 as might be in communication with DCD 105 of FIG. 1. HH monitor/alert device 106 comprises processor 201 coupled to: entry device 202 (such as a keypad or touch display), display 203 (such as an LCD), microphone/speaker 204 (with necessary operational circuitry not shown in the figure), and wireless communication module 205. Processor 201 might be employed to generally process or otherwise implement software algorithms to control various functions and circuitry of HH monitor/alert device 106, such as, but not limited to, i) receiving information from one or more of entry device 202, microphone of microphone/speaker 204, and wireless communication module 205, ii) processing the information, and iii) providing the processed information to one or more of display 203, speaker of microphone/speaker 204, and wireless communication module 205. Wireless communication module 205 coordinates communication through WCL to DCD 105 through a predefined link standard and/or protocol.

Returning to FIG. 1, subject to preprogrammed rules, DCD 105 might collect vital sign (VS) data from a patient and generate a VS signal containing the VS data in a convenient format for display and or interpretation by a caregiver. Also subject to preprogrammed rules, DCD 105 might also examine information provided from each monitoring device 102. DCD 105 continuously reviews and charts the information from patient sensors 101 using descriptive statistics (dynamic mode). Data might be processed by a control methodology using a statistical “control chart” subject to rules, such as the Western Electric Rules of Statistical Quality Control (WE-SQC) described in, for example, the AT&T Statistical Quality Control Handbook published by the Western Electric Company. Rules governing out-of-control situations when applied to data plotted on a chart within control limits are called “Western Electric Rules.” Associated with the control methodology, “control limits” might have predefined boundaries. When monitored data reaches one or more of these control limits, appropriate alerting signals are transmitted through WCL to HH monitor/alert device 106. Upon receipt of the alerting signal, HH monitor/alert device 106 might i) display the data associated with reaching the control limit and ii) provide an alarm (audible or vibration) to alert the caregiver of the event.

DCD 105 is coupled to central medical database 107, WE-SQC rule and procedure algorithm (WRPA) module 108, and user control interface 109. Central medical database 107 might be employed to store patient information collected from monitoring devices 102(1) through 102(n), as well as descriptive statistical information as processed by DCD 105. In addition, central medical database 107 might provide data about patients that are monitored and statistical and/or medical information employed by algorithms used by DCD 105. Such information contained in central medical database 107 is termed herein as “vital sign, patient care, and medical treatment statistical information.” Algorithms used by DCD 105 might be received from a database of algorithms in WRPA module 108. WRPA module 108 might be employed to tailor known algorithms to statistical monitoring of data specific to a patient through access of information in central medical database 107. User control interface 109 is employed to program or otherwise predefine actions or operation of DCD 105, central medical database 107, and WRPA module 108.

Statistical rules of quality control might help the caregiver or doctor monitor the patient's vital signs and other critical responses whenever necessary since the rules might hasten detection of health issues before it may worsen the patient's condition. The rules for statistical quality control can be applied to monitoring data points in charts. While the rules are best applied in a manufacturing environment, these rules are applied in accordance with embodiments of the present invention to control charts with patient data. Several methods of collecting data and plotting charts from the gathered data are well-known such as, for example, plotting a data chart using a “moving range” chart as appropriate. Since classical methods of passive data collection that are prevalent in the statistical quality control are not available to the caregiver and doctors to set up control limits for patients, some empirical studies and observations may be necessary to determine chart limits. These limits may then be widened or narrowed as warranted by the individual patient vital sign behavior, age, medical and other health conditions.

For example, it is generally accepted that the systolic blood pressure for adult males of 40 years should be between 90 to 140 mm Hg. Mild hypertension range is 140 to 160 mm Hg, moderate range is 160 to 200 mm Hg and beyond 200 mm Hg is severe. Such ranges can be used to plot the mean (X-bar) and the range on the control chart may be selected from the recommended guidelines. Analogous limits may be set for diastolic pressure, where the corresponding numbers are 90 to 100 mm Hg, 100 to 120 mm Hg and above 120 mm Hg. Thus “warning”, “critical” and “danger” limits on the control charts can be assigned, with the methods described herein being activated when data-points fall within these zones.

Different criteria can be used to determine when a data point on the chart is out-of-control. A pattern in any of the responses of patient data is critical to monitoring out-of-control situations. With normal statistical variations, the pattern will not show any unusual behavior. This behavior might vary from simple situations where one can observe stratification (eight or more points on the same side of the mean), out-of-control situations resulting in a warning event, a point outside of the control limits, four out of five consecutive points outside of the first set of limits, etc.

Statistical monitoring of data provides at least the following advantages. First, statistical monitoring tracks moving averages and applies statistical methods to detect trends in the data or other forms of patient health behavior. Second, warning signs or approaching danger conditions might be deduced from the statistical trends, providing early alerts and reports to caregivers. Third, a caregiver might regularly remotely access patient monitoring data according to a statistical rule, allowing the caregiver to check on the patient more or less often as required. Fourth, statistical monitoring might advantageously detect when sensors or other forms of patient monitoring equipment are malfunctioning or otherwise improperly performing.

FIG. 3 shows an exemplary graph of data points versus thresholds as might be employed by the data collection device of FIG. 1 in accordance with statistical quality control. As shown in FIG. 3, for a given sensor data, such as oxygen level, DCD 105 collects data points for which mean value 301 is tracked. Based on predefined rules for the patient case, three control limits, “Warning”, “Critical”, and “Danger” are defined. “Warning”, “Critical”, and “Danger” limits might be defined based on standard deviation quantities above or below the mean value. As shown, when data points 302 and 303 occur, they are outside the “Warning” limit, and an alert is generated by DCD 105 that is sent to HH monitor/alert device 106.

Further embodiments of the present invention might employ HH monitor/alert device 106 in connection with an ambulance or other form of patient transport. DCD 104 in the patient transport might collect information from monitoring devices coupled to patient sensors, and then optionally encrypt the information. The collected information is then transmitted to HH monitor/alert device 106 at the medical facility to alert the caregiver to the patient's vital signs. Such information might be transmitted from the patient transport through a wireless or cellular telephone network, or through a mobile broadband connection.

Embodiments of the present invention might provide the following advantages. Communication of alerts or other signals to a patient caregiver generally reduces response time to a situation, improving the success rate for a given intervention. Further, a caregiver might monitor certain vital signs of a patient remotely in a location with ease, rather than returning to the patient's location, thereby increasing a level of care provided to the patient.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The present invention may be implemented as circuit-based processes, including possible implementation as a single integrated circuit (such as an ASIC or an FPGA), a multi-chip module, a single card, or a multi-card circuit pack. As would be apparent to one skilled in the art, various functions of circuit elements may also be implemented as processing blocks in a software program. Such software may be employed in, for example, a digital signal processor, micro-controller, or general-purpose computer.

The present invention can be embodied in the form of methods and apparatuses for practicing those methods. The present invention can also be embodied in the form of program code embodied in tangible media, such as magnetic recording media, optical recording media, solid state memory, floppy diskettes, CD-ROMs, hard drives, or any other machine-readable storage medium, wherein, when the program code is loaded into and executed by a machine, such as a computer, the machine becomes an apparatus for practicing the invention. The present invention can also be embodied in the form of program code, for example, whether stored in a storage medium, loaded into and/or executed by a machine, or transmitted over some transmission medium or carrier, such as over electrical wiring or cabling, through fiber optics, or via electromagnetic radiation, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits. The present invention can also be embodied in the form of a bitstream or other sequence of signal values electrically or optically transmitted through a medium, stored magnetic-field variations in a magnetic recording medium, etc., generated using a method and/or an apparatus of the present invention.

As used herein in reference to an element and a standard, the term_compatible_means that the element communicates with other elements in a manner wholly or partially specified by the standard, and would be recognized by other elements as sufficiently capable of communicating with the other elements in the manner specified by the standard. The compatible element does not need to operate internally in a manner specified by the standard.

Unless explicitly stated otherwise, each numerical value and range should be interpreted as being approximate as if the word “about” or “approximately” preceded the value of the value or range.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A patient monitoring system comprising:
a data collection device (DCD) coupled to one or more monitoring devices through corresponding links, the DCD configured to i) collect signals from one or more monitoring devices, wherein each signal represents patient data collected by one or more sensors coupled to a patient, and ii) process each signal in accordance with one or more algorithms, wherein said DCD generates at least one of a vital sign (VS) signal and an alerting signal for each signal based on the one or more algorithms for irregular behavior or an out-of-bound event with the corresponding signal; and
a portable communication device (PCD), in wireless communication with the DCD, configured to i) receive the VS signal and the alerting signal and ii) provide information associated with the VS signal and the alerting signal to a caregiver.

2. The invention of claim 1, further comprising:
a medical database, coupled to the DCD, having medical data relating to at least one of vital sign, patient care, and medical treatment statistical information and adapted to receive patient data from the DCD;
a rule and procedure algorithm processor, coupled to at least one of the DCD and the medical database, configured to i) receive a portion of the medical data from the medical database associated with a patient, ii) receive a portion of the patient data, and iii) process the portion of medical data and the portion of patient data in accordance with a statistical monitoring algorithm having a control methodology.

3. The invention of claim 2, wherein the rule and procedure algorithm processor implements a control methodology as a control chart based on a set of rules.

4. The invention of claim 3, wherein the set of rules is a Western Electric Rules of Statistical Quality Control (WE-SQC).

5. The invention of claim 1, wherein the PCD comprises:
a processor;
a display;
a speaker module, and
a wireless communication module,
wherein the processor is:
i) coupled to the display, the speaker module, and the wireless communication module,
ii) configured to receive at least one of a vital sign (VS) signal and an alerting signal from the wireless communication module, and
iii) configured to provide information associated with the VS signal and the alerting signal to a caregiver through at least one of the display and the speaker module.

6. The invention of claim 5, further comprising an entry device, wherein:
the entry device is at least one of a keypad and touch display; and
the processor receives caregiver queries from the entry device.

7. The invention of claim 6, wherein:
the processor is configured to i) communicate the caregiver queries from the entry device to the DCD, receive answers to the caregiver queries from the DCD, and communicate the answers to the caregiver through at least one of the speaker module and the display.

8. The invention of claim 5, wherein the wireless communication module implements a wireless communication link operating in accordance with at least one of a WiFi (802.11), WiMax (802.16), personal area network (PAN) (802.15) families of standards, low frequency radio, or 2.5 and 3G wireless telephony standards.

9. The invention of claim 1, wherein the wireless communication between the PCD and the DCD operates in accordance with at least one of a WiFi (802.11), WiMax (802.16), personal area network (PAN) (802.15) families of standards, low frequency radio, or 2.5 and 3G wireless telephony standards.

10. A portable communication device (PCD) for a patient monitoring system comprising:
a processor;
a display;
a speaker module, and
a wireless communication module,
wherein the processor is:
i) coupled to the display, the speaker module, and the wireless communication module,
ii) configured to receive at least one of a VS signal and an alerting signal from the wireless communication module, and iii) configured to provide information associated with the VS signal and the alerting signal to a caregiver through at least one of the display and the speaker module.

11. The PCD for a patient monitoring system according to claim 10:
wherein the PCD is in wireless communication with a data collection device (DCD) coupled to one or more monitoring devices through corresponding links, the DCD configured to i) collect signals from one or more monitoring devices, wherein each signal represents patient data collected by one or more sensors coupled to a patient, and ii) process each signal in accordance with one or more algorithms, wherein said DCD generates at least one of the VS signal and the alerting signal for each signal based on the one or more algorithms for irregular behavior or an out-of-bound event with the corresponding signal.

12. A method of patient monitoring comprising:
collecting, by a data collection device (DCD) coupled to one or more monitoring devices through corresponding links, signals from one or more monitoring devices, wherein each signal represents patient data collected by one or more sensors coupled to a patient;
processing each signal in accordance with one or more algorithms;
generating, by the DCD, at least one of a vital sign (VS) signal and an alerting signal for each signal based on the one or more algorithms for irregular behavior or an out-of-bound event with the corresponding signal;
receiving, by a portable communication device (PCD) in wireless communication with the DCD, the VS signal and the alerting signal; and
providing, by the PCD, information associated with the VS signal and the alerting signal to a caregiver.

13. The invention of claim 12, further comprising:
receiving, by a medical database coupled to the DCD and having medical data relating to at least one of vital sign, patient care, and medical treatment statistical information, patient data from the DCD;
receiving, by a rule and procedure algorithm processor coupled to at least one of the DCD and the medical database, i) a portion of the medical data from the medical database associated with a patient, and ii) a portion of the patient data; and
processing, by the rule and procedure algorithm processor, the portion of medical data and the portion of patient data in accordance with a statistical monitoring algorithm having a control methodology.

14. The invention of claim 13, wherein the step of processing each signal in accordance with one or more algorithms implements a control methodology as a control chart based on a set of rules.

15. The invention of claim 14, wherein the set of rules is a Western Electric Rules of Statistical Quality Control (WE-SQC).

16. The invention of claim 12, further comprising:
transmitting, by the PCD, caregiver queries from an entry device of the PCD to the DCD;
receiving, by the PCD, answers to the caregiver queries from the DCD; and
communicating, by the PCD, the answers to the caregiver through at least one of a speaker module and a display.

17. The invention of claim 16, wherein the steps of transmitting and receiving, by the PCD, comprise the steps of implementing a wireless communication link and the operating wireless communication link in accordance with at least one of a WiFi (802.11), WiMax (802.16), personal area network (PAN) (802.15) families of standards, low frequency radio, or 2.5 and 3G wireless telephony standards.

18. A machine-readable storage medium, having encoded thereon program code, wherein, when the program code is executed by a machine, the machine implements a method for patient monitoring, comprising the steps of:
collecting, by a data collection device (DCD) coupled to one or more monitoring devices through corresponding links, signals from one or more monitoring devices, wherein each signal represents patient data collected by one or more sensors coupled to a patient;
processing each signal in accordance with one or more algorithms;
generating, by the DCD, at least one of a vital sign (VS) signal and an alerting signal for each signal based on the one or more algorithms for irregular behavior or an out-of-bound event with the corresponding signal;
receiving, by a portable communication device (PCD) in wireless communication with the DCD, the VS signal and the alerting signal; and
providing, by the PCD, information associated with the VS signal and the alerting signal to a caregiver.

19. The invention of claim 18, further comprising:
receiving, by a medical database coupled to the DCD and having medical data relating to at least one of vital sign, patient care, and medical treatment statistical information, patient data from the DCD;
receiving, by a rule and procedure algorithm processor coupled to at least one of the DCD and the medical database, i) a portion of the medical data from the medical database associated with a patient, and ii) a portion of the patient data; and
processing, by the rule and procedure algorithm processor, the portion of medical data and the portion of patient data in accordance with a statistical monitoring algorithm having a control methodology.

20. The invention of claim 19, wherein the step of processing each signal in accordance with one or more algorithms implements a control methodology as a control chart based on a set of rules.
